(19) United States
(12) Patent Application Publication
(10) Pub. No.: US 2008/0199972 A1
(43) Pub. Date: Aug. 21, 2008

(54) Spectroscopic Method For the Detection of Analytes

(76) Inventor: Frank Sellrie, Berlin (DE)

Correspondence Address: J C PATENTS, INC., 4 VENTURE, SUITE 250, IRVINE, CA, 92618, US

(21) Appl. No.: 11/913,547

(22) PCT Filed: May 2, 2006
(86) PCT No.: PCT/DE06/00756

§ 371 Date: Nov. 2, 2007

(30) Foreign Application Priority Data
May 2, 2005 (DE) 10 2005 020 384.1

(51) Int. Cl. G01N 33/566; C07K 16/00; C07K 14/54

(52) U.S. Cl. 436/501; 530/391.5; 530/351

(57) ABSTRACT

The present invention relates to methods for the detection of one or more analytes, in particular pathogens, viruses, prions, bacteria, parasites, pharmaceuticals, antibiotics, cytostatics, psychoactive substances, narcotics, analgesics, cardiac drugs, metabolites, coagulation inhibitors, hormones, interleukins and cytokines, performance-enhancing drugs, drugs, toxins, noxious substances, pesticides, insecticides, wood preservatives, herbicides, fungicides, explosives, vitamins and flavors by providing a conjugate of the analyte and an europium cryptate fluorophore or respectively a terbium cryptate fluorophore together with an antibody which is specific for the analyte and an antibody which is specific for the europium cryptate fluorophore or respectively the terbium cryptate fluorophore and by spectroscopically determining the fluorescence quenching which occurs when the analyte is added. Furthermore the present invention relates to conjugates of the analyte and the europium cryptate fluorophore or respectively terbium cryptate fluorophore as well as to a kit for the detection of analytes.

The present invention relates to methods for the detection of one or more analytes by providing a conjugate of the analyte and an europium (or terbium) cryptate fluorophore together with a defined quantity of antibodies which are specific for the analyte and a defined quantity of antibodies which are specific for the europium (terbium) cryptate fluorophore and by spectroscopically determining the fluorescence quenching which occurs when the analyte is added. Furthermore, the present invention relates to conjugates of the analyte and the europium (terbium) cryptate fluorophore as well as to a kit containing the components required for the detection of the one or more analytes.

Various methods using specific antibodies for the detection of different analytes are known. For example, EP 0 343 346 B describes a fluorescence immunoassay. U.S. Pat. No. 4,261,968 describes a method for the detection of an analyte by using antibodies and fluorescence quenchers, wherein the degree of fluorescence quenching directly correlates with the amount of analytes to be determined.

The present invention aims at providing a simple method which can be universally applied for the detection of any substances, as well as at providing suitable compounds for said method.

Said aim is achieved by the technical teaching of the independent claims of the present invention. Further advantageous embodiments of the invention result from the dependent claims, the description and the examples.

The present invention relates to a method for the detection of one or more analytes, wherein the method comprises the following steps:
a) providing the following three components
   a conjugate of the analyte and an europium cryptate fluorophore,
   an antibody specific for the analyte and
   an antibody specific for the europium cryptate fluorophore,
   wherein the conjugate of the analyte and a europium cryptate fluorophore only allows for one single antibody to be bound at the same time,
b) addition of the analyte and
c) spectroscopic measurement of the fluorescence of the europium cryptate fluorophores.

Also, Terbium may be used instead of the europium, so that any specification given herein is valid both for europium and terbium. Thus, the present invention relates to a method for the detection of one or more analytes, wherein the method comprises the following steps:
a) providing the following three components
   a conjugate of the analyte and a terbium cryptate fluorophore,
   an antibody specific for the analyte and
   an antibody specific for the terbium cryptate fluorophore,
   wherein the conjugate of the analyte and a terbium cryptate fluorophore only allows for one single antibody to be bound at the same time,
b) addition of the analyte and
c) spectroscopic measurement of the fluorescence of the terbium cryptate fluorophores.

Three components are required for the method according to the invention: firstly a conjugate of the substance to be detected, i.e. the analyte, and a fluorophore and secondly an antibody which is specific for the substance to be found and thirdly, a second antibody which is specific for the fluorophore. Hydrogen peroxide is a fourth preferred component. The function of said component is described in greater detail below. The analyte, the presence of which or the concentration of which is to be determined, may be considered an additional component. Said component, however, is not mandatory, as the assay according to the invention may also be used for detecting the absence of a certain analyte.

The term “fluorescence” or “fluorescence quenching” as used herein is intended to describe any luminescence effect; consequently it includes phosphorescence so that the terms “fluorescence” or respectively “fluorescence quenching” also comprise “phosphorescence” or respectively “phosphorescence quenching”.

Generally, all current types of antibodies may be used, such as for example polyclonal antibodies, monoclonal antibodies, humanized antibodies, human antibodies, chimeric antibodies, recombinant antibodies, bispecific antibodies as well as antibody fragments, wherein monoclonal antibodies are preferred.

Antibody fragments, aptamers, mimotopes, fab fragments, fc fragments, peptides, lectins, nucleotides, peptidomimetics, gapmers, ribozymes, CpG-oligomers, DNAzymes, riboswitches or lipids may be used instead of antibodies.

Methods for producing said compounds, especially antibodies and monoclonal antibodies, are known to the one skilled in the art.

Preferably, the antibodies which are specific for the analyte (analyte specific antibodies) and the antibodies which are specific for the conjugate of analyte and fluorophore (fluorophore specific antibodies) are used in a defined and known ratio. The term analyte specific antibody describes an antibody which specifically binds to the analyte, wherein, with respect to said bond, it is irrelevant whether the analyte is completely or only partially bound by the antibody or whether a part of the linker and/or of the fluorophore is also bound by the antibody or whether even the linker and/or the fluorophore is/are completely involved in the bond. The only important aspect consists in the fact that the binding has to take place in such way that neither a second analyte specific antibody nor a fluorophore specific antibody are able to bind. Similarly, with respect to the fluorophore specific antibody, it is of no relevance whether said antibody binds the fluorophore completely or only partially and whether the linker and/or the analyte is/are completely or partially involved in the bond. The fluorophore specific antibody, too, has to be bound in such way that neither an analyte specific antibody nor a further fluorophore specific antibody are capable of binding. Thus, the type of antibody bond is not essential to the invention, as long as no other antibody can be bound at the same time.

The analyte/fluorophore conjugate is designed such that only one single antibody can bind thereto at the same time. Consequently, binding of two analyte specific antibodies or two fluorophore specific antibodies or one analyte specific antibody and one fluorophore specific antibody at the same time is excluded.

Furthermore, the invention is based on the principle that the fluorescence of the analyte fluorophore conjugate is not significantly affected by the binding of the analyte specific antibody, while binding of the fluorophore specific antibody leads to the fluorescence being quenched or at least in the fluorescence being measurably reduced.

If a solution of said three components, that is conjugate and analyte specific antibody and fluorophore specific antibody is provided, the conjugate in the solution is bound by one of the two antibodies; consequently, it is for example bound to an extent of 50% by the analyte specific antibody and to an extent of about 50% by the fluorophore specific antibody. A small quantity is present in non-bound form.

If a sample solution containing the substance to be detected, i.e. the analyte, is added to said solution, the analyte and the conjugate containing the analyte compete for the binding site at the analyte specific antibody. Consequently, a part of the analyte specific antibody bound to the conjugate releases the conjugate and binds to the substance to be detected, i.e. the analyte. Said procedure leads to an increase in the concentration of free conjugate, which, however, is immediately bound by the fluorophore specific antibody, present in excess in the solution. Due to the binding of the fluorophore specific antibody, the fluorescence is quenched, a fact which can be spectroscopically measured.

A reduction of the fluorescence of the solution indicates the presence of the substance to be detected, the analyte, wherein the degree of fluorescence quenching sheds light on the quantity of analyte in the sample solution.

Thus, the inventive methods are based on the general principle of establishing a connection between the reduction of the fluorescence and the presence and concentration of a certain analyte.

Evidently, it will be apparent to the one skilled in the art that, in lieu of the fluorescence quenching antibodies, such antibodies may be used which enhance or measurably increase the fluorescence of the fluorophore if bound to an europium cryptate fluorophore (or respectively terbium cryptate fluorophore) or if bound to a conjugate of analyte and europium cryptate fluorophore (or respectively terbium cryptate fluorophore). The expression “measurably increase” describes an increase which can be distinctly determined by means of spectroscopic methods and distinguished from background fluorescence. In said inventive methods, the degree of the increase in fluorescence and the concentration of the analyte to be measured are correlated.

Thus, the present invention relates in particular to antibodies being capable of binding to a europium cryptate fluorophore (or respectively terbium cryptate fluorophore) and/or a conjugate of the analyte and a europium cryptate fluorophore (or respectively a terbium cryptate fluorophore) and being capable of quenching or measurably reducing the fluorescence of the fluorophore due to said bond.

On the other hand, the present invention also relates to antibodies being capable of binding to a europium cryptate fluorophore (or respectively to terbium cryptate fluorophore) and/or a conjugate of the analyte and a europium cryptate fluorophore (or respectively a terbium cryptate fluorophore) and of enhancing or measurably increasing the fluorescence of the fluorophore thanks to said bond.

The preferably used europium cryptate fluorophores (or respectively terbium cryptate fluorophores) are those described herein.

The inventive antibodies are produced according to standard methods, e.g. by means of hybridoma techniques (Köhler and Milstein, Nature 1975, 256, 495-497), by using Epstein Barr viruses, Xeno mice or by means of cells which are permanently viable in cell culture and which produce antibodies (Pasqualini and Arap, Proc. Natl. Acad. Sci. USA 2004, 6, 257-259). Furthermore, the antibodies may be derived from recombinant antibody libraries produced, for example, by means of phage display or ribosome display or similar techniques.

Antibodies reducing the fluorescence of chelate bound or cryptate bound lanthanides, particularly of europium (Eu³⁺) and terbium (Tb³⁺) or such antibodies enhancing the fluorescence of chelate-bound or cryptate-bound lanthanides, particularly of europium (Eu³⁺) and Terbium (Tb³⁺), may be used for establishing various measurement systems. In particular, this concerns their application in screening procedures for antibody-producing cells of desired specificity and the determination of antibody affinities in relation to the binding affinity of a known antibody. Similarly, such antibodies may be used for the development of immunoassays based on fluorescence resonance energy transfer (FRET). The application described herein is directed on the use of the aforementioned antibodies in a homogenous assay in solution. Evidently, such antibodies in solid phase assays, particularly in the form of a microarray, can also be used, just as the inventive antibodies when real time PCR systems are realized.

The following samples may be particularly used as sample solutions: saliva samples, blood samples, urine samples, mucus samples, digested tissue samples, water samples, wastewater samples, samples from chemical production processes, extracted soil samples, food samples, samples from biological and biochemical production processes, fermentation solutions and the like. Serum samples as well as diluted samples of the aforementioned kind or diluted extracts of, for example, soil samples, mucus samples, fermentation solutions and the like are particularly preferred. The preparation of suitably diluted solutions for spectroscopic analysis methods will be apparent to the one skilled in the art.

Any analyte against which antibodies can be produced and which may be bound to a europium cryptate fluorophore (or respectively terbium cryptate fluorophore) can be used as substance to be detected. A covalent bond, possibly via a linker of the analyte, to the europium cryptate fluorophore (or respectively to the terbium cryptate fluorophore) is preferred. The linker is situated between the analyte and europium cryptate fluorophore (or respectively the terbium cryptate fluorophore) and can consist of any chemical components or fragments which increase the distance between the analyte and the europium cryptate fluorophore (or respectively the terbium cryptate fluorophore), wherein the distance must not be so large that, due to the distance between the analyte and europium cryptate fluorophore (or respectively terbium cryptate fluorophore), the simultaneous binding of an analyte specific antibody and a fluorophore specific antibody will be possible.

Examples of possible analytes include:
Analytes from human diagnostics:

### Pathogens:

viruses: e.g. measles, influenza, SARS, HIV, hepatitis, herpes, tick-born meningoencephalitis, etc.
Prions
Bacteria: e.g. tuberculosis, Lyme disease, cholera, pertussis, etc.
Parasites: e.g. malaria, leishmania, etc.
Tumor markers
   Examples of tumor markers include:

beta 2 microglobulin, ferritin, p53, corticotropin (ACTH), growth hormone (GH, hGH), prolactin (PRL), chorionic gonadotropin (hCG and CG), calcitonin (CT), thyroglobulin (tg), carcinoembryonic antigen (CEA), CA antigens 125, 72-4, 15-3 and 19-9, alpha-fetoprotein (AFP), prostate specific antigen (PSA), galactosyl transferase 11.

Examples of pathogens include:

brucella IgG and IgM; chlamydia pneumonia IgA, IgG and IgM; chlamydia trachomatis IgA, IgG and IgM; CMV IgG and IgM; Dengue virus IgG and IgM; EBV-VCA IgA, IgG and IgM; H. pylori IgA, IgG and IgM; HSV 1+2 IgG and IgM; measles IgG and IgM; mumps IgG and IgM; mycoplasma IgG and IgM; rubella IgG and IgM; salmonella IgG and IgM; toxoplasma IgA, IgG and IgM; treponima pallidum IgG and IgM; VZV IgG and IgM; hepatitis A, B, C; HIV1, 2.

### Pharmaceuticals: e.g.

Antibiotics (ampicillin, amoxicillin, penicillin, etc.)
Cytostatics (doxurubicin, cisplatin, etc.)
Psychoactive substances
Narcotics
Analgesics
Cardiac drugs
Coagulation inhibitors

### Metabolites:

e.g. hormones (steroid hormones, gonadotropins, etc.)
Interleukins and cytokines, etc.
Triiodothyronines
Plasma proteins
Enzymes (secretases, lipases, phosphatases, etc.)
Lipids (cholesterol, etc.)
Disease factors (autoimmune diagnostics, etc.)
   Targets in sports medicine:
performance-enhancing drugs (amphetamines, growth hormones, erythropoietin, etc.)
   Drugs e.g. heroin, cocaine, LSD, etc.
   Toxins mycotoxins, bacterial toxins: cholera toxin, pertussis toxin, etc.
   Examples of antibiotics, antimycotics and antiviral drugs include:

acyclovir, ampicillin, amoxicillin, aminoglycosides, amphotericin B, azithromycin, cefazolin, cefepime, cefotaxime, cefotetan, cefpodoxime, ceftazidime, ceftizoxime, ceftriaxone, cefuroxime, cephalexin, chloramphenicol, clotrimazole, ciprofloxacin, clarithromycin, clindamycin, dapsone, dicloxacillin, doxycycline, erythromycin lactobionate, fluconazole, foscarnet, ganciclovir, gatifloxacin, imipenem/cilastatin, isoniazid, itraconazole, ketoconazole, metronidazole, nafcillin, nitrofurantoin, nystatin, pentamidine, penicillin, piperacillin/tazobactam, rifampin, quinupristin/dalfopristin, ticarcillin/clavulanate, trimethoprim sulfamethoxazole, valacyclovir, vancomycin

Examples of hormones include:

ACTH, C-peptide, cortisol, DHEA-S, estradiol, estriol, testosterone, insulin, proinsulin, progestrone, PTH.

Examples of interleukins, cytokines and growth factors include:

erythropoietin (EPO), human growth hormone (hGH), interleukin 1 to 18, tumor necrosis factor-α,β (TNF-α, β), fibroblast growth factor (FGF), granulocyte, macrophage, granulocyte/macrophage colony stimulating factor (G-CSF, M-CSF, GM-CSF), vascular endothelial growth factor (VEGF), platelet growth factor (PGF), nitrogen oxides, leukotrienes.

Examples of plasma proteins and other plasma molecules include

C-reactive protein (CRP), albumin, coagulation factors, complement factors, immunoglobulins (IgA, IgE, IgG, IgM), phosoholipases, vasopressin, homocystein, myoglobin, troponin 1, caspases, insulin, blood group determination, rhesus factor, alpha amylase, cholinesterase, creatine kinase, gamma-GT, GOT/ASAT, GPT/ALAT, LDH, pancreatic lipase, phosphatases, bilirubin, glucose, creatinine, lipoproteins.

Examples of autoimmune diagnostics include:

ACA IgG, ANA Screen IgG, cardiolipin IgA, cardiolipin IgG, cardiolipin IgM, cardiolipin total ab, dsDNA IgG, mitochondrial (MA) ab, RNP/Sm IgG, Scl-70, Sm IgG, SSA IgG, SSB IgG, thyroglobulin (tg) ab, thyroid peroxidase (TPO) IgG

Targets in sports medicine:
Performance-enhancing drugs (amphetamines, growth hormones, erythropoietin, etc.)
   Drugs e.g. heroin, cocaine, LSD, etc and degradation products thereof.
   Toxins mycotoxins, bacterial toxins: cholera toxin, pertussis toxin, etc.
   Examples of performance-enhancing drugs include:

anabolic agents, e.g. bolasterone, boldenone, dehydrochloromethyltestosterone, dihydrotestosterone, clostebol, fluoxymesterone, mesterolone, metandienone, metenolone, methyltestosterone, nandrolone, norethandrolone, oxandrolone, oxymesterone, oxymetholone; stanozolol, testosterone and chemically or pharmacologically related compounds; beta-2 agonists (e.g. clenbuterol); amphetamines: e.g. amineptine, amphetamine, amphetaminil, benzphetamine, dimethylamphetamine, ethylamphetamine, fenetylline, fenproporex, furfenorex, mesocarb, methoxyphenamine, methylamphetamin, methylphenidate, morazone, pemoline, phendimetrazine, pipradol, pyrovalerone and chemically or pharmacologically related compounds; corticosteroids for oral, intramuscular or intravenous administration; peptide hormones and analogs thereof: e.g. chorionic gonadotropin HCG (human sex hormone), corticotrophin (ACTH), growth hormone (HGH, somatotropin), erythropoietin (EPO). Any corresponding factors released from the substances mentioned above are equally forbidden.

Stimulants: e.g. amiphenazole, coffein, cathin, chlorphentermine, clobenzorex, clorprenaline, cropropamide, crotethamide, ephedrine, etafedrine, etamivan, fencamfamin, mefenorex, methylephedrine, nikethamide, pentetrazole, phenylpropanolamine, prolintane, propylhexedrine, strychnine and chemically or pharmacologically related compounds.

Narcotics/analgesics: e.g. alphaprodine, anileridine, buprenorphine, dextromoramide, dextropropoxyphen, diamorphine, dipipanone, ethoheptazine, ethylmorphine, levorphanol, methadone, morphine, nalbuphine, pentazocine, pethidine, trimeperidine and chemically or pharmacologically related compounds.

Examples of drugs include:

amphetamines, barbiturates, benzodiazepines, cannabinoids, cocaine metabolite, cotinine, fentanyl, flunitrazepam, LSD, methamphetamine, morphine, opiates, PCP, tricyclics

Analytes from veterinary medicine:

pathogens, pharmaceuticals, metabolites, performance-enhancing drugs, toxins, etc.

Analytes from environmental diagnostics (air, water and soil analyses):
Noxious substances:
pesticides (PCB, atrazine, etc.)
Insecticides (pyrethroids, bacillus thuringiensis toxin etc.)
Wood preservatives (Lindan, etc.)
Herbicides (diuron, monuron, etc.)
Fungicides
Toxins and explosives (e.g. dioxin, TNT, etc.)
Colors and varnishes
Environmental pollutants, toxic substances
   Examples of colors and varnishes include:

phthalates, azo dyes, glycol ethers, glycol esters, dioxins.

Examples of insecticides include:

dimethoate, deltamethrin, diflubenzuron, alpha-, zeta-cypermethrin, fenoxycarb, tebufenpyrad, pirimicarb, azadirachtin, piperonyl butoxide, pyrethrine, flocoumafen, chlorpyrifos, permethrin, chalcogran, decadiene carboxylic acid methyl ester, ipsdienol, (S)-cis-verbenol, methylbutenol

Examples of pesticides include:

3,4,5-trimetacarb, 3-hydroxycarbofuran, 5-hydroxy-clethodim sulfone, 5-hydroxy imidacloprid, 5-hydroxy thiabendazole, 6-chloro-3-phenyl-pyridazine-4-(pyridate metabolite), acephate, acetamiprid, acibenzolar-5-methyl, aclonifen, acrinathrin, alachlor, aldicarb, aldicarb sulfoxide, aldoxycarb, ametryn, amidosulfuron, aminocarb, anilazine, atrazine, avermectin B1a, avermectin B1b, azamethiphos, azinphos-ethyl, azinphos-methyl, azoxystrobin, benalaxyl, bendiocarb, benfuracarb, bensulfuron methyl, benzoximate, bifenox, bitertanol, boscalid, bromacil, bromuconazole, bupirimate, buprofezin, butocarboxim, butocarboxim sulfoxide, butoxycarboxim, buturon, carbaryl, carbendazim, carbetamides, carbofuran, carbosulfan, carboxin, carfentrazone-ethyl, chlorbromuron, chlorfenvinphos, chlorfluazuron, chloridazon, chlorotoluron, chloroxuron, chlorpyrifos, chlorsulfuron, chlorthiophos, cinosulfuron, clethodim, clethodim imine sulfone, clethodim imine sulfoxide, clethodim sulfone, clethodim sulfoxide, clodinafop-propargyl, clofentezine, clomazone, clopyralid, cloquintocet-mexyl, coumaphos, cyanazine, cyanofenphos, cyazofamid, cycloate, cymoxanil, cyprodinil, cyromazine, daminozide, deltamethrin, demeton-s-methyl, demeton-s-methyl-sulfone, desmedipham, desmethylformamido-pirimicarb, desmethyl-pirimicarb, dialifos, di-allate, diazinon, dichlofluanid, dichlorvos, diclobutrazol, diethofencarb, difenoconazole, difenoxuron, diflufenican, dimefuron, dimethachlor, dimethenamid, dimethoate, dimethomorph, diniconazole, diphenylamine, disulfoton, diuron, dodemorph, EPN, epoxiconazole, ethiofencarb, ethiofencarb sulfone, ethiofencarb sulfoxide, ethion, ethirimol, ethofumesate, ethoprophos, etofenprox, etrimfos, famoxadone, fenamiphos, fenarimol, fenazaquin, fenbuconazole, fenfuram, fenhexamid, fenoxaprop-ethyl, fenoxycarb, fenpiclonil, fenpropathrin, fenpropidin, fenpropimorph, fenpyroximate, fenthion, fenuron, flamprop-isopropyl, flamprop-methyl, flazasulfuron, florasulam, fluazifop-butyl, flufenacet, flufenoxuron, fluometuron, fluoroglycofene-ethyl, fluquinconazole, fluridone, fluoroxypyr-meptyl, flurtamone, flusilazole, flutriafol, folpet, fonofos, fosthiazate, fuberidazole, furathiocarb, haloxyfop-etotyl, haloxyfop-methyl, heptenophos, hexaconazole, hexazinone, hexythiazox, imazalil, imidacloprid, indoxacarb, iodosulfuron-methyl, iprodione, iprovalicarb, isazophos, isofenphos, isoproturon, isoxathion, kresoxim-methyl, linuron, malaoxon, malathion, MCPA-butotyl, mecarbam, mepanipyrim, metalaxyl, metamitron, metazachlor, metconazole, methabenzthiazuron, methamidophos, methfuroxam, methidathion, methiocarb, methomyl, methoxyfenozide, metobromuron, metolachlor, metosulam, metoxuron, metribuzin, metsulfuron-methyl, molinate, monocrotophos, monolinuron, monuron, myclobutanil, naled, napropamides, neburon, nicosulfuron, nicotine, nuarimol, ofurace, omethoate, oxadixyl, oxamyl, oxamyl-oxime, oxycarboxin, oxydemeton-methyl, paclobutrazol, paraoxon-methyl, parathion, parathion-methyl, penconazole, pencycuron, pendimethalin, phenmedipham, phenthoate, phorate, phorate sulfoxide, phosalone, phosmet, phosphamidon, phoxim, picolinafen, picoxystrobin, pirimicarb, pirimiphos-ethyl, pirimiphos-methyl, primisulfuron-methyl, prochloraz, procymidone, profenofos, promecarb, prometryne, propachlor, propamocarb, propargite, propetamphos, propham, propiconazole, propoxur, propyzamide, prosulfocarb, prosulfuron, prothiofos, pymetrozine, pyraclostrobin, pyrazophos, pyridaben, pyridaphenthion, pyridate, pyrimethanil, pyriproxyfen, quinalphos, quinmerac, quinoclamine, quinoxyfen, quizalofop-ethyl, rimsulfuron, rotenone, simazine, simetryn, spiroxamine, sulfosulfuron, sulfotep, sulprofos, tebuconazole, tebufenozide, tebufenpyrad, tebutam, tebuthiuron, terbacil, terbufos, terbumeton, terbuthylazine, terbutryn, tetrachlorvinphos, tetraconazole, thiabendazole, thiacloprid, thiamethoxam, thifensulfuron-methyl, thiodicarb, thiofanox, thiofanox sulfone, thiofanox sulfoxide, thiophanate (-ethyl), thiophanate-methyl, tolclofos-methyl, tolylfluanid, triadimefon, triadimenol, triasulfuron, triazophos, tribenuron-methyl, trichlorfon, tricyclazole, trietazine, trifloxystrobin, triflumizole, triflusulfuron-methyl, triticonazole, vamidothion.

Examples of noxious substances and toxic substances include:

1,1,1-trichloroethane, 1,1,2,2-tetrachloroethane, 1,1,2-trichloroethane, 1,1-dichloroethane, 1,1-dichloroethene, 1,2,3,4,6,7,8,9-octachlorodibenzofuran, 1,2,3,4,6,7,8-heptachlorodibenzo-p-dioxin, 1,2,3-trichlorobenzene, 1,2,3-trichloropropane, 1,2,4-trichlorobenzene, 1,2-dibromo-3-chloropropane, 1,2-dibromoethane, 1,2-dichlorobenzene, 1,2-dichloroethane, trans-1,2-dichloroethene, 1,2-dichloroethylene, 1,2-diphenylhydrazine, 1,3,5-trinitrobenzene, 1,3-butadiene, 1,3-dichlorobenzene, cis- and trans-3-dichloropropene, 1,4-dichlorobenzene, 2,3,4,7,8-pentachlorodibenzofuran, 2,3,5,6-tetrachlorophenol, 2,3,7,8-tetrachlorodibenzofuran, 2,3,7,8-tetrachlorodibenzo-p-dioxin, 2,4,5-trichlorophenol, 2,4,6-trichlorophenol, 2,4,6-trinitrotoluene, 2,4-dichlorophenol, 2,4-dimethylphenol, 2,4-dinitrophenol, 2,4-dinitrotoluene, 2,6-dinitrotoluene, 2-butanone, 2-chloroaniline, 2-chlorophenol, 2-hexanone, 2-methylnaphthalene, 3,3′-dichlorobenzidine, 4,4′-methylenebis(2-chloroaniline), 4,6-dinitro-o-cresol, 4-aminobiphenyl, 4-nitrophenol, acenaphthene, acetone, acrolein, aldrin, alpha-chlordene, amosite asbestos, aroclor 1016, 1221, 1232, 1240, 1242, 1248, 1254, 1260, 1262, arsenic acid, asbestos, benzene, benzidine, benzo(a)anthracene, benzo(a)pyrene, benzo(b or k)fluoranthene, benzofluoranthene, benzopyrene, bis(2-chloroethyl)ether, bis(2-ethylhexyl)adipate, bis(2-methoxyethyl) phthalate, bromodichloroethane, bromoform, butyl benzyl phthalate, butylate, calcium arsenate, carbazole, carbon tetrachloride, carbophenothion, chlordane, chlordecone, chlorobenzene, chlorodibromomethane, chloroethane, chloromethane, chlorpyrifos, chrysene, chrysotile asbestos, cis-chlordane, coal tar creosote, para-cresol, cresols, cyanide, cyclotrimethylenetrinitramine (RDX), o,p′- or p,p′-ddd, p,p′-dde, o,p′- or p,p′-ddt, di(2-ethylhexyl)phthalate, diazinon, dibenzo(a,h)anthracene, (chlorinated) dibenzofuran, dibenzothiophene, dibromochloropropane, dichlorobenzene, dichloroethane, dichloroprop, dichlorvos, dicofol, dieldrin, dimethoate, dimethyl formamide, dimethylarsinic acid, di-n-butyl phthalate, dinitrotoluene, disulfoton, (alpha-; beta-) endosulfan (sulfate), endrin (aldehyde; ketone), ethoprop, ethyl ether, ethylbenzene, fluoranthene, formaldehyde, gamma-chlordene, guthion, heptachlor, heptachlor epoxide, heptachlorobiphenyl, heptachlorodibenzofuran, heptachlorodibenzo-p-dioxin, hexachlorobenzene, hexachlorobutadiene, alpha-, beta-, delta-, gamma-hexachlorocyclohexane, hexachlorocyclopentadiene, hexachlorodibenzofuran, hexachlorodibenzo-p-dioxin, hexachloroethane, hydrazine, hydrogen cyanide, indeno(1,2,3-cd)pyrene, methoxychlor, methyl isobutyl ketone, methyl parathion, methylene chloride, methylmercury, naled, naphthalene, n-nitrosodimethylamine, n-nitrosodi-n-propylamine, n-nitrosodiphenylamine, ortho-cresol, oxychlordane, parathion, pentachlorobenzene, pentachlorobiphenyl, pentachlorobutadiene, pentachlorodibenzofuran, pentachlorodibenzo-p-dioxin, pentachlorophenol, phenanthrene, phenol, phorate, polonium-210, polybrominated biphenyls, polychlorinated biphenyls, polycyclic aromatic hydrocarbons, p-xylene, pyrene, pyrethrum, s,s,s-tributyl phosphorotrithioate, strobane, styrene, tetrachlorobiphenyl, tetrachlorodibenzo-p-dioxin, tetrachloroethane, tetrachloroethylene, tetrachlorophenol, thiocyanate, toluene, toxaphene, trans-chlordane, tributyltin, trichlorobenzene, trichloroethane, trichloroethylene, trichlorofluoroethane, trifluralin, vinyl chloride.

Analytes from agriculture and forestry:

plant and animal pathogens, mycotoxins, environmental pollutants, etc.

Analytes in process engineering/food technology/biotechnology/cosmetics:

chemical and biotechnological synthesis products (inclusively primary products, intermediate products, byproducts and degradation products, such as vitamins, flavors, pharmaceuticals, toxins, lipids, amino acids, glycerides, carbohydrates, fibers, sweeteners, colorants, preservative agents, polysaccharides, mycotoxins, etc.

Examples of food additives include:

acesulfam K, acetylated oxidated starch, acetylated starch, acetylated distarch adipate, acetylated distarch phosphate, allura red AC, alpha tocopherol, aluminum lacquers, aluminum sodium sulfate, amaranth, ammonia caramel coloring, ammonia sulfite caramel coloring, annatto, anthocyans, malic acid, ascorbinic acid, aspartame, azorubine, beetroot red, bentonite, benzoic acid, benzyl alcohol, succinic acid, beta-apo-8′-carotenal, beta-apo-8′-carotinic acid ethyl ester, beta-cyclodextrin, beeswax (white and yellow), biphenyl, bixin, boric acid, brown FK, brown HAT, brilliant blue FCF, brilliant black BN, butadiene-styrene copolymer, butylhydroxyanisole (BHA), butylated hydroxytoluoene (BHT), calcium 5′-ribonucleotide, canthaxanthin, capsanthin, capsorubin, carbamide, carboxymethylcellulose, carnauba wax, carotenes (mixed carotenes, beta carotene), carrageenan, cellulose (microcrystalline cellulose, cellulose powder), quinoline yellow, chlorophylls, chlorophyllins, cochenille red A, enzymatically hydrolyzed carboxymethylcellulose, erythrosin, gamma tocopherol, sunset yellow S, gellan, green S, hydroxypropyl cellulose, hydroxypropyl distarch phosphate, hydroxypropylmethyl cellulose, hydroxypropyl starch, indigotin 1, invertase, copper-containing complexes of the chlorophylls, copper-containing complexes of the chlorophyllins, curcumin, monostarch phosphate, sodium carboxymethyl cellulose, neohesperidin DC, nisin, norbixin, oxydized starch, patent blue V, pectins (pectin, amidated pectin), phosphatized distarch phosphate, phosphoric acid, polydextrose, polyethylene glycol 6000, polyethylene wax oxidates, polyvinyl esters of the non-branched fatty acids C2-C18, polyvinylpolypyrrolidone, polyvinylpyrrolidone, riboflavin, riboflavin 5′-phosphate, red 2G, saccharin and Na-, K- and Ca-salts thereof (saccharin, saccharin sodium, saccharin calcium, saccharin potassium), sucrose acetate isobutyrate, sorbitan monolaurate, sorbitan monooleate, sorbitan monopalmitate, sorbitan monostearate, sorbitan tristearate, starch sodium octenylsuccinate, tannin, tartrazine, thaumatin, tragacanth, processed eucheuma seaweed, linked sodium carboxymethylcellulose, xanthan.

Examples from cosmetics include:

PEG and PEG derivatives, formaldehyde resins, musk compounds, tensides, paraffins, aromatic amines, methyl-, butyl-, ethyl-, propyl-, isobutyl parabens, poloxamer 184, hydrolyzed elastine, allantoin, lactic acid enzymes, xanthan.

In literature, various chelates with various cations are known as fluorophores. Even proteins or antibodies with a certain amount of aromatic groups may serve as fluorophores. Especially europium cryptate fluorophores and terbium cryptate fluorophores were shown to be very advantageous with respect to the present invention, as said fluorophores have a characteristic luminescence spectrum which can be very well separated from the possible fluorescence of other components, such as proteins present in the sample to be measured. Europium cryptate fluorophore and terbium cryptate fluorophore are distinguished by a light absorption caused by the organic part of the cryptate and by the direct energy transfer to the europium ion. The europium ion is present in oxidation state 3+. The terbium ion, too, is present in oxidation state 3+. The europium cryptate fluorophore is preferably excited at a wave length of 337 nm by means of a nitrogen laser. Emission is measured at 620 nm (and at 545 nm in the case of terbium). Fluorescence of the europium cryptate fluorophores and terbium cryptate fluorophores is significantly longer compared to that of other fluorophores or other fluorescent components of the measured sample, so that the fluorescence of the europium cryptate fluorophores or respectively terbium cryptate fluorophores can be well and accurately determined despite a possible background fluorescence caused by other components or other fluorophores.

In addition to the so called “small molecules”, that is small chemical molecules such as pharmacological ingredients or toxins which can be bound directly or via a linker to a europium cryptate fluorophore (or respectively to a terbium cryptate fluorophore); it is also possible to bind macromolecules to a europium cryptate fluorophore (or respectively to a terbium cryptate fluorophore). The fluorophore specific antibodies also bind to conjugates consisting of a macromolecule and a europium cryptate fluorophore (or respectively terbium cryptate fluorophore) so that the methods described herein may also be used for the detection of macromolecules.

Macromolecules to be mentioned include in particular polynucleotides, oligonucleotides, DNA fragments, RNA fragments, proteins, glycoproteins, lipoproteins, polysaccharides, DNA, RNA, mimotopes, fab fragments, fc fragments, peptides, lectins, peptidomimetics, gapmers, ribozymes, CpG oligomers, DNAzymes, riboswitches, polymers, hormones, vitamins, carbohydrates, glycerides or lipids.

A preferred embodiment of the present invention uses conjugates between an analyte molecule and a europium cryptate fluorophore molecule or respectively complex. According to the invention it is also possible to bind two molecules from the same analyte to a europium cryptate fluorophore molecule or to bind respectively one molecule from two different analytes to the europium cryptate fluorophore. On the other hand, it is also possible that two identical or even different europium cryptate fluorophore molecules are bound to an analyte molecule. This may be advantageous for increasing the detection accuracy of a certain analyte or for allowing the simultaneous detection of several analytes. Furthermore, it will be apparent to the one skilled in the art to provide conjugates of more than two analyte molecules and/or europium cryptate fluorophore molecules, such as for example conjugates of three identical or different analyte molecules and one or two or more europium cryptate fluorophore molecules. This is also true for terbium.

Preferred cryptates used are cyclic and particularly bicyclic compounds. It is further preferred that the cryptates have nitrogen bridgehead atoms. Furthermore, cryptates preferably contain one or more pyridine residues or pyridine components and bipyridine residues or respectively bipyridine components or bipyridine fragments are particularly preferred. Thus, bicyclic cryptates having two nitrogen bridgehead atoms are particularly preferred.

Furthermore, pyridine components are preferably present in a cyclic, particularly in a bicyclic system. Preferred cryptates have the following general formula:

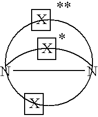

wherein [symbol]
independently from each other represent an alkyl residue, alkene residue, aryl residue, heteroaryl residue, alkylaryl residue, heteroarylalkyl residue, cyclyl residue, heterocyclyl residue and particularly comprise a methylene group, ethylene group, ehthyleneoxy group, ethenylene group, alkylene amino group, alkyleneoxy group, phenyl group, biphenyl group, pyridyl group, bipyridyl group, benzopyridyl group, benzo bipyridyl group, imidazole group, pyrrolinyl group and/or pyrrolidinyl group.

Cycles having a ring of the size of 16 to 20 atoms, preferably 17 to 19 atoms are particularly preferred. In this context, ring size means that only those atoms forming the shortest ring in proximity to the europium or respectively terbium are considered. Cyclic and particularly bicyclic ring systems having rings in the aforementioned size are particularly well suited for the incorporation of euorpium³⁺ (or respectively Tb³⁺). Tris-bipyridyl cryptates, pyridyl dibypyirdyl cryptates as well as dipyridyl bipyridyl cryptates, i.e. cryptates having 6, 5, or 4 pyridyl rings are particularly preferred.

Below, some examples of potential cryptates with or without Eu³⁺ or respectively Tb³⁺ are shown, wherein Tb³⁺ can be used instead of Eu³⁺.

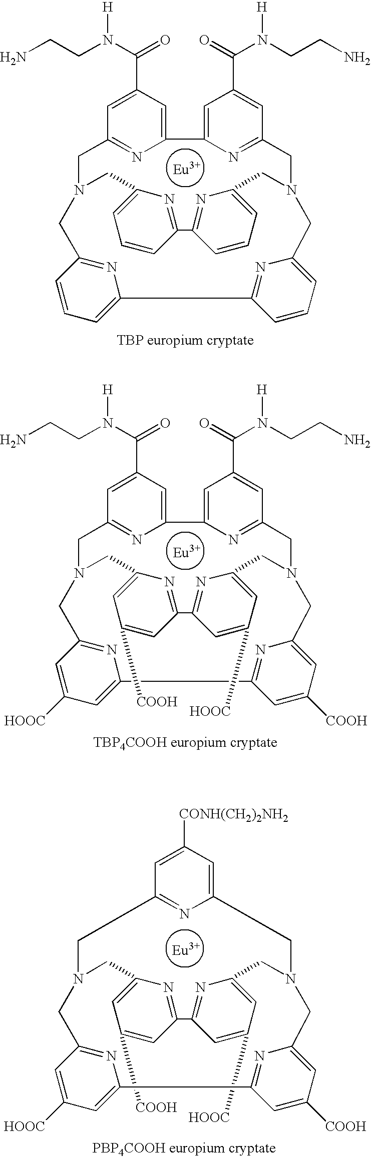

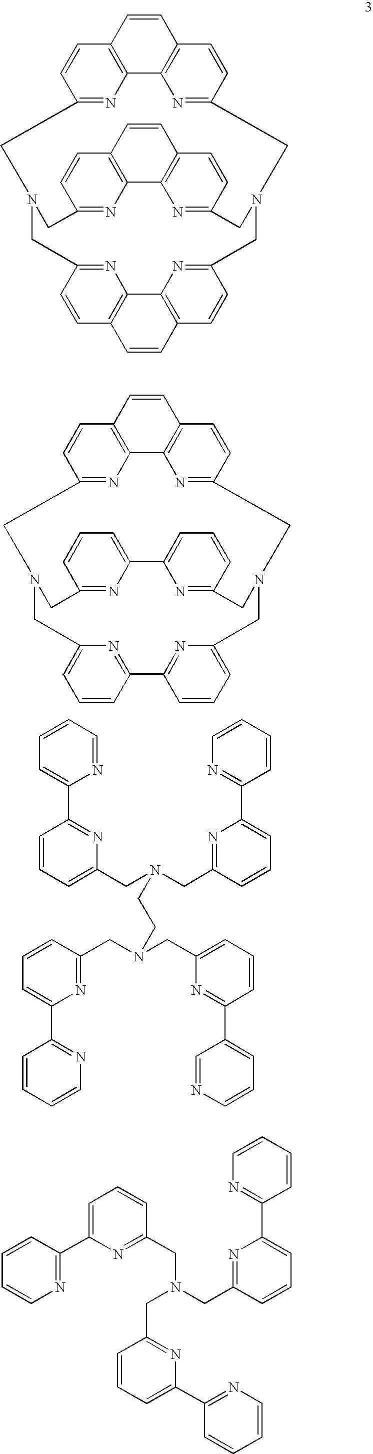

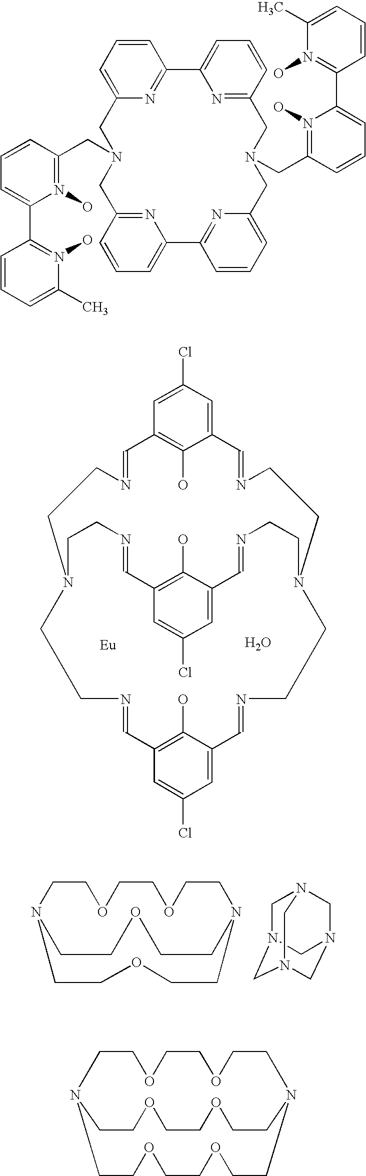

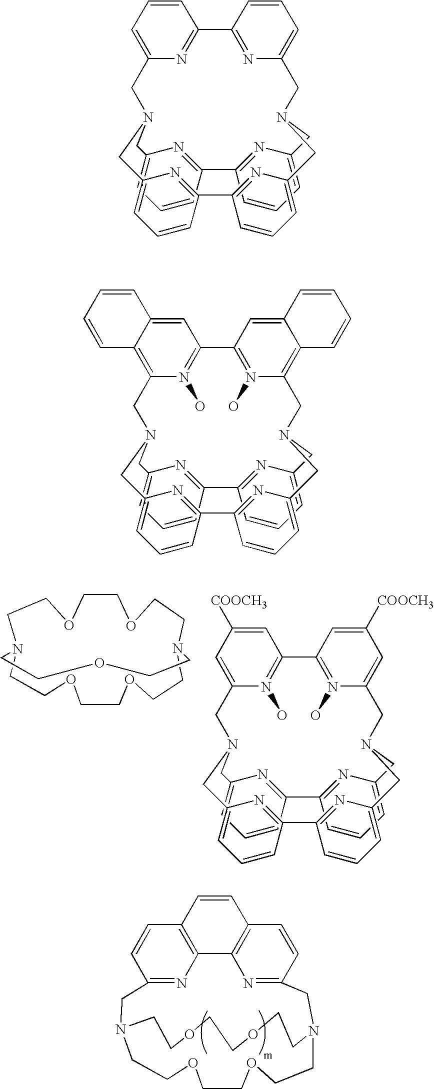

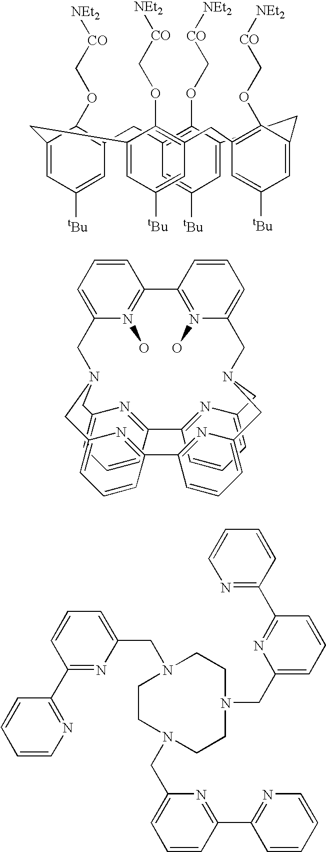

The analyte can be bound to the cryptate by means of ionic, covalent or lipophilic interaction, by intercalation or incorporation as well as via hydrogen bonds, wherein the covalent bond is preferred.

Thus, it is also preferred that the cryptates carry a functional group at least one ring, to which group the analyte may be bound directly or indirectly via a linker.

The following are particularly suitable functional groups: halogens, hydroxy groups, carboxylate group, carbonyl group, thiol group, amino group, cyanate group, isocyanate group. Linkage to the analyte is preferably performed by means of an imine bond, amide bond, ester bond, ether bond, aldol addition, ketal bond, acetal bond, nucleophilic substitution, carbonate bond, urethane bond, thioester bond, thioether bond or urea bond.

The following structures illustrate preferred examples

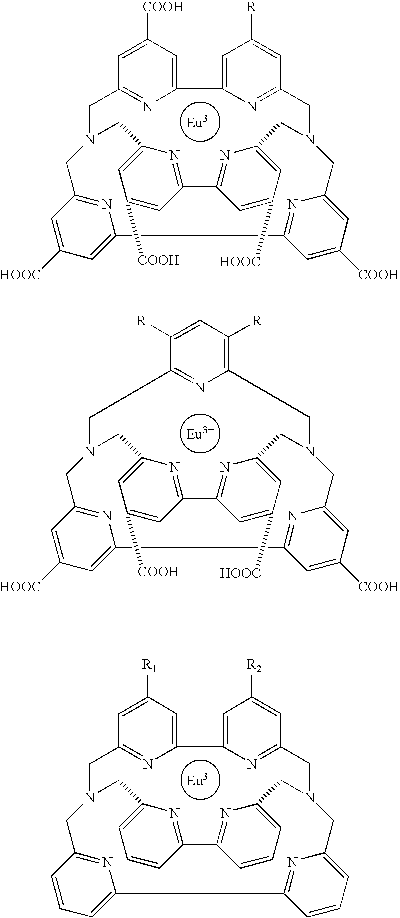

wherein the carboxyl group or the residues R represent potential binding sites for a molecule of the analyte.

Preferably, only one single molecule of the substance to be detected is bound to a molecule of the europium cryptate fluorophore (or respectively of the terbium cryptate fluorophore). Furthermore, it is essential to the invention that only one single antibody is bound per molecule of the conjugate consisting of europium cryptate fluorophore (or respectively terbium cryptate fluorophore) and the analyte. Thus, potentially used linkers must not be of such a length that the distance between europium cryptate fluorophore (or respectively terbium cryptate fluorophore) and analyte becomes sufficient for a fluorophore specific antibody and an analyte specific antibody to bind. Thanks to steric effects, the binding of two antibodies to one conjugate is excluded. The substances mentioned above may be bound to the europium cryptate fluorophore (or respectively to the terbium cryptate fluorophore) as analytes.

If used together with the cryptates mentioned herein, Eu³⁺ and Tb³⁺ allow for a time-resolved measurement of the fluorescence (TRF: time resolved fluorescence) and thus for a clear distinction between measured and background signal.

Another embodiment of the invention aims at detecting antibodies formed by an organism and directed against a certain analyte instead of detecting the analyte itself. In said embodiment, a conjugate of analyte and europium cryptate fluorophore (or respectively terbium cryptate fluorophore) is used in combination with a fluorophore specific antibody and the increase in fluorescence is determined when the sample solution is added since the analyte specific antibody to be detected at least partially displaces the fluorophore specific antibody in the compound with the conjugate as the simultaneous binding of analyte specific antibody and fluorophore specific antibody is excluded.

The methods described herein allow for a qualitative and in particular quantitative measurement of the analytes. Furthermore, the methods according to the invention are advantageous in that all conventional immunoassays (in particular all heterogeneous immunoassays, such as ELISA) may be adapted to the methods according to the invention. Consequently, work processes would be considerably facilitated as the methods according to the invention represent homogenous assays, i.e. users only have to mix their sample with the measurement sample and subsequently determine its fluorescence. Furthermore, the process can be easily automatised, e.g. in routine diagnostics, by means of which both time and money may be advantageously saved.

Moreover, it is particularly preferred that a certain amount of hydrogen peroxide (H₂O₂) be added to the sample solution containing the conjugate of europium cryptate fluorophore (or respectively terbium cryptate fluorophore) and analyte as well as the analyte specific antibody and the fluorophore specific antibody. It has been shown that hydrogen peroxide has positive influences on the fluorescence, i.e. the effects of luminescence. In particular, hydrogen peroxide seems to increase or respectively intensify luminescence, which results in a significant increase in the measuring sensitivity or respectively allows for very small quantities of analyte to be measured. The mode of action of hydrogen peroxide, however, is not clear. Preferably at least one equimolar quantity, with respect to the conjugate, of hydrogen peroxide is added. Higher quantities of hydrogen peroxide, such as a 2-fold molar excess (2 mol equivalents), a 3-fold molar excess (3 mol equivalents), a 5-fold molar excess (5 mol equivalents) or a 10-fold molar excess (10 mol equivalents) with respect to the conjugate are equally possible. Therefore, the hydrogen peroxide may be used in a molar ratio with respect to the conjugate of from 0.1:1 to 50:1, preferably from 0.5:1 to 10:1, further preferred from 0.8:1 to 4:1 and particularly preferred from 1:1 to 2 (H₂O₂):1 (conjugate).

Another preferred embodiment of the present invention comprises the addition of another luminophore to the sample solution form the analyte specific antibody and the fluorophore specific antibody as well as from the conjugate of europium cryptate fluorophore (or respectively terbium cryptate fluorophore) and the analyte. This additional luminophore serves for transferring or respectively receiving fluorescence (luminescence) signals by means of FRET (fluorescence resonance energy transfer), so that different analytes can be analyzed in a measuring sample. Said additional luminophore or said additional luminophores preferably are so called quantum dots of different wavelengths. Such a composition consisting of the conjugate, the two antibodies and the quantum dots allows for a multiplex screening of several different analytes in one measuring sample.

Besides, the present invention is directed to a composition comprising:
a) a conjugate of the analyte and a europium cryptate fluorophore, (or respectively terbium cryptate fluorophore)
   wherein the conjugate of the analyte and a europium cryptate fluorophore, (or respectively terbium cryptate fluorophore) only allows for one single antibody to be bound at the same time,
b) an antibody specific for the analyte and
c) an antibody specific for the europium cryptate fluorophore (or respectively terbium cryptate fluorophore)

Said composition may further contain hydrogen peroxide and/or additional luminophores as an additional component.

Such a composition is an essential component of a kit for the detection of various analytes. Such kit contains at least one of the aforementioned compositions for the detection of one or more analytes and one or more sample vessels for performing the reaction as well as an instruction on how the reaction is to be carried out.

Furthermore, a composition may also contain different conjugates of different analytes with the same or with different europium cryptate fluorophores (or respectively terbium cryptate fluorophores) and different analyte specific antibodies in order to simultaneously detect the presence of different analytes.

The method according to the invention relates to the realization of a homogenous immunoassay. In a homogenous system, the measuring sample (containing a plurality of other components, of which type, concentration and influence on the measuring system are largely unknown and the analyte) and the measuring system (containing the components for generating the measuring signal) are combined. Directly after that, the measurement is performed, i.e. all components of the measuring sample (even those which unspecifically influence and consequently adulterate the measuring system and thus the measuring signal) remain in the system. Said interferences can only be controlled as long as the measurements take place in a defined matrix (e.g. an aqueous solution of the analyte). Such systems, however, lack practical relevance. For practically relevant measurements (e.g. in serum material of patients, production residues or digestions of soil samples) the measuring signal and the background signal have to be distinguished. Only if these prerequisites are fulfilled, the advantages of the homogenous system with respect to the simplicity of the measuring process and the resulting reduction of time and work will become apparent.

The method according to the invention is a homogenous fluorescence quenching immunoassay. The contexts described before are also valid for this system. Systems using “simple” fluorophores (such as fluorescein) have been known for about 20 years—but have never gained practical relevance due to the reasons described, despite the indisputable function of the system and its obvious advantages. The problem which had not been resolved so far consisted in the matrix effects described.

In the mixture of measuring sample and measuring system not only the fluorophore but also a plurality of other components from the measuring sample (e.g. serum proteins) is excited such that they become fluorescent. In this context, light emission in said fluorophores takes place in the same time window as the emission of the components remaining in the measuring mixture of the measuring sample. The type and strength of the background signal depend on the individual composition of the measuring sample and cannot be determined beforehand. As a result, measurements of practically relevant measuring samples (such as patient sera) cannot be carried out.

However, such problems can be resolved by means of time-resolved fluorescence measurement (TRF). For said purpose, the fluorophores used so far are displaced by chelates or respectively cryptates of the elements terbium and europium. Due to the radiation free energy transitions within said complex the emission of light is delayed. As described before, the components of the measuring sample as well as the fluorophore are excited such that they become fluorescent at the same time. The measuring signal, however, can be detected once the background fluorescence has faded. Thus, practically relevant samples may also be analyzed. This is made possible by the combination of the homogenous testing principle with a time-resolved measurement of the fluorescence.

The method according to the invention is therefore particularly suited for measuring samples which include a plurality of other components, such as blood samples or soil samples, without a previous purification of the measuring sample being required.

### EXAMPLES

### Example 1

### 1.) Preparation of an Immune Serum Against TBP Europium Cryptate

Immunization of a balb/c mouse with:
50 μg of streptavidin—TBP europium cryptate 1^{st} immunization
25 μg of streptavidin—TBP europium cryptate 2^{nd} immunization (after one month)
blood draw and serum extraction (known to the expert)

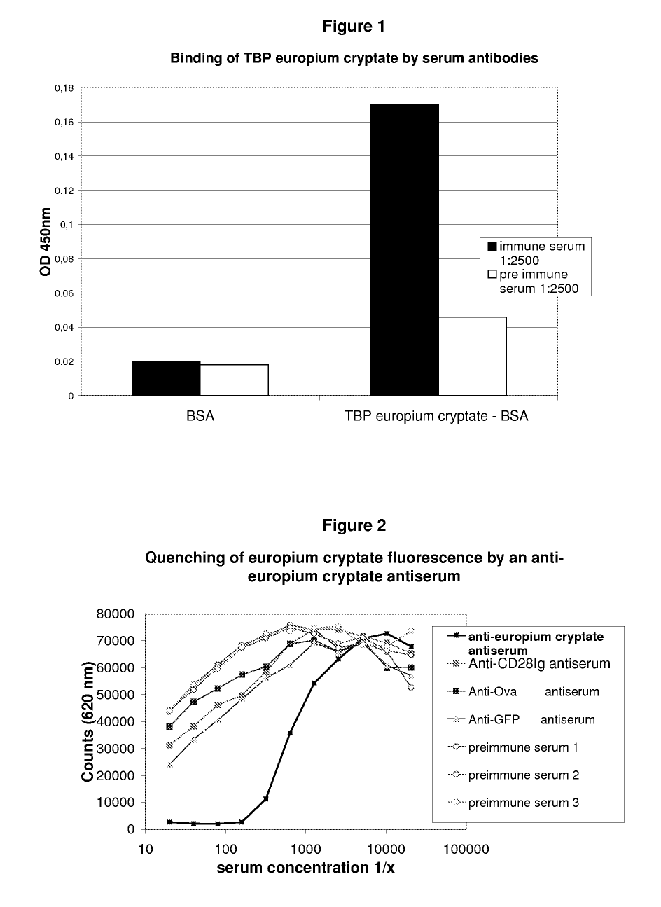

### 2.) Detection of the Binding of TBP Europium Cryptate by the Serum (FIG. 1)

Preparation of a test conjugate:
Coupling of TBP europium cryptate to BSA by linking free amino groups by means of glutaraldehyde according to methods known to the one skilled in the art.
Detection of antibody binding to BSA conjugate in ELISA.

### 3.) Detection of Fluorescence Quenching Due to Antibodies Binding to Tbp Europium Cryptate (FIG. 2)

Measuring sample (150 μl) consisting of:

| | | |
|---|---|---|
| TBP europium cryptate | 1:200000 (final concentration) | 75 μl |
| immune serum against | | |
| TBP europium cryptate | dilution series | 75 μl |
| or respectively | | |
| three preimmune sera (circles) | | |
| three immune sera against three | | |
| other antigens (squares) | | |

Fluorescence measurement:
excitation 337 nm; emission 620 nm:

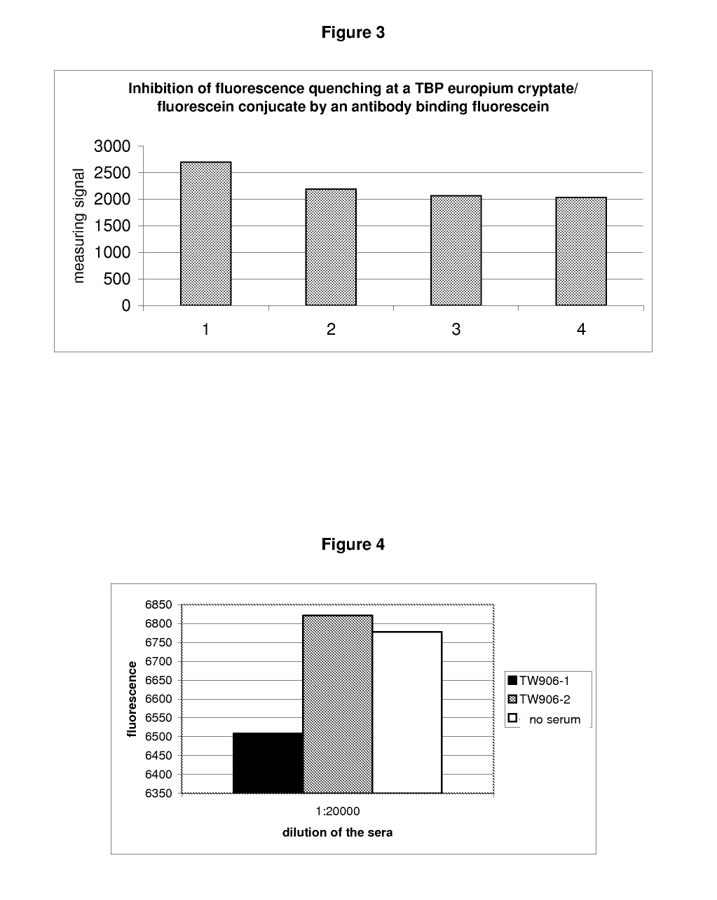

### 4.) Detection of the Displacement of the Fluorophore-Binding Antibody by the Analyte-Binding Antibody (FIG. 3)

Preparation of a conjugate of analyte (fluorescein) and fluorophore (europium cryptate)
Fluorescein isothiocyanates (FITC) and TBP europium cryptate (0.03 mM) were reacted in a molar ratio of 2:1. PBS was used as a medium and the reaction time was 4 hours at room temperature.
Measuring sample (150 μl) consisting of:

| | | |
|---|---|---|
| Conjugate | 1:200000 (final concentration) | 75 μl |
| Immune serum against TBP | | |
| europium cryptate | 1:200 (final concentration) | 37.5 μl |
| antibody DE1 (anti-fluorescein | | 37.5 μl |
| antibody) or respectively | | |
| antibody D35 | | |
| (anti-glucose-oxidase | | |
| antibody) | | |

Fluorescence measurement:
excitation 337 nm; emission 620 nm

FIG. 3 displays the measured values of the measuring samples nos. 1 to 4.

| Measuring | | | | Measuring |
|---|---|---|---|---|
| sample No | Fluorophore | Serum | Antibody | Signal |
| 1 | TBP europium | anti-TBP europium | anti-fluorescein | 2700 |
| | cryptate/fluorescein | cryptate | | |
| 2 | TBP europium | anti-TBP europium | anti-galactosidase | 2191 |
| | cryptate/fluorescein | cryptate | | |
| 3 | TBP europium cryptate | anti-TBP europium | anti-fluorescein | 2065 |
| | | cryptate | | |
| 4 | TBP europium cryptate | anti-TBP europium | anti-galactosidase | 2035 |
| | | cryptate | | |
| 5 | TBP europium cryptate | preimmune serum | anti-fluorescein | 8894 |
| 6 | none | none | none | 578 |

### Example 2

### Experiments with Respect to Europium Chelate

### Objective:

Detection of polyclonal antibodies quenching the fluorescence of europium chelates in serum samples.

### Model System:

Test animals (balb/c mice) were immunized with europium chelate/OVA conjugates (OVA=ovalbumin). The serum samples prepared were tested for their characteristic of quenching the fluorescence of an europium chelate/BSA conjugate. The europium chelate used was [EuL] having the following structure, wherein Ln represents europium:

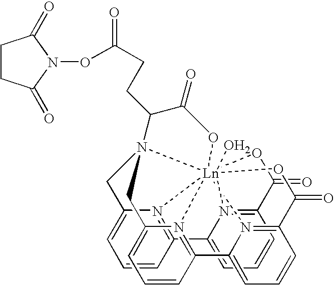

### Preparation of the Europium Chelate/Protein Conjugate

The europium chelate [EuL] was present in form of NHS ester. Coupling to the carrier proteins BSA (bovine serum albumin) and OVA took place overnight at room temperature in phosphate buffer, pH 7.4.

Subsequent to the coupling, the conjugate was dialyzed against PBS and used in the test without previous purification or characterization.

Immunization of the Test Animals with [EuL]-OVA and Serum Extraction 2 Balb/c Mice:

The first immunization was carried out with 100 μg of conjugate in complete Freund's adjuvant. The second immunization took place after one month with 50 μg of the conjugate without the adjuvant. Blood was drawn one week after the 2^{nd} immunization.

### Test of the Immune Sera for Quenching the Fluorescence of an [EuL]-BSA Conjugate

The measuring sample is composed of 75 μl of the diluted serum samples and 75 μl of [EuL]/BSA conjugate (dilution 1:10000; final concentration). All components of the measuring sample were diluted in PBS-NKS (5% calf serum in PBS).

The fluorescence was measured after one hour in the cryptor (Cezanne). The excitation was caused by means of a nitrogen laser at 337 nm; the time-resolved measurement of the fluorescence was realized at 620 nm.

### Result:

A TW906-1 serum (serum from the immunization 906-1) showed the desired fluorescence-quenching effect, i.e. antibodies were present in the serum which antibodies quenched (in analogy to the cryptate) the fluorescence of europium chelates (see FIG. 4).

### Example 3

### Experiments with Respect to the Haptene Europium Cryptate

### Objective:

Detection of the displacement of a monoclonal antibody quenching the fluorescence of europium from its bond to a conjugate of haptene (small molecule) and fluorophore (europium cryptate) by means of a second molecule binding the haptene in the conjugate as well as use of said effect for determining the concentration of the haptene.

### Model System:

A conjugate of europium cryptate (fluorophore) and biotin (vitamin H) (haptene, analyte) was used. The monoclonal antibody G24-BA9 which quenches the fluorescence of europium cryptates was displaced from its bond to the fluorophore by the protein streptavidin binding biotin. Said effect could be inversed by external administration of the free analyte (biotin). The fluorescence changes occurring in this context allow for the determination of the concentration of the free analyte (biotin).

### Preparation of the Europium Cryptate/Biotin (Vitamin H) Conjugate Eu(NH₂)-Biotin

### Materials:

| | | |
|---|---|---|
| Cryptate NH₂ | (cisbio) | (cryptate NH₂ = TBP europium cryptate, see |
| | | page 18) |
| Biotin NHS | (Fluka) | |

### Coupling:

Biotin NHS and cryptate NH₂ were predissolved in DMSO. Both components were mixed in a molar ratio of 7 (cryptate) to 1 (biotin) in a coupling reaction. Coupling took place overnight at room temperature in a 0.1 M carbonate buffer, pH 9.0.

The Eu(NH₂) biotin conjugate was used without the conjugate being further purified.

### Determination of the Biotin (Vitamin H) Concentration in the Fluorescence Quenching Immunoassay

The following four components were mixed in measuring reactions of respectively 150 μl in the specified final concentrations:

| | | |
|---|---|---|
| Cryptate | Eu-NH₂-biotin | dilution 1:300000 |
| Quencher | G24-BA9 | 20 μg/ml (cryptate antibody) |
| displacing antibody | streptavidin | 3 μg/ml |
| Analyte | biotin | dilution series 0 to 100 ng/ml |

All components of the measuring sample were diluted in PBS-NKS (5% calf serum in PBS).

The fluorescence was measured after one hour in the cryptor (Cezanne). The excitation was caused by means of a nitrogen laser at 337 nm; the time resolved measurement of the fluorescence was carried out at 620 nm.

### Result:

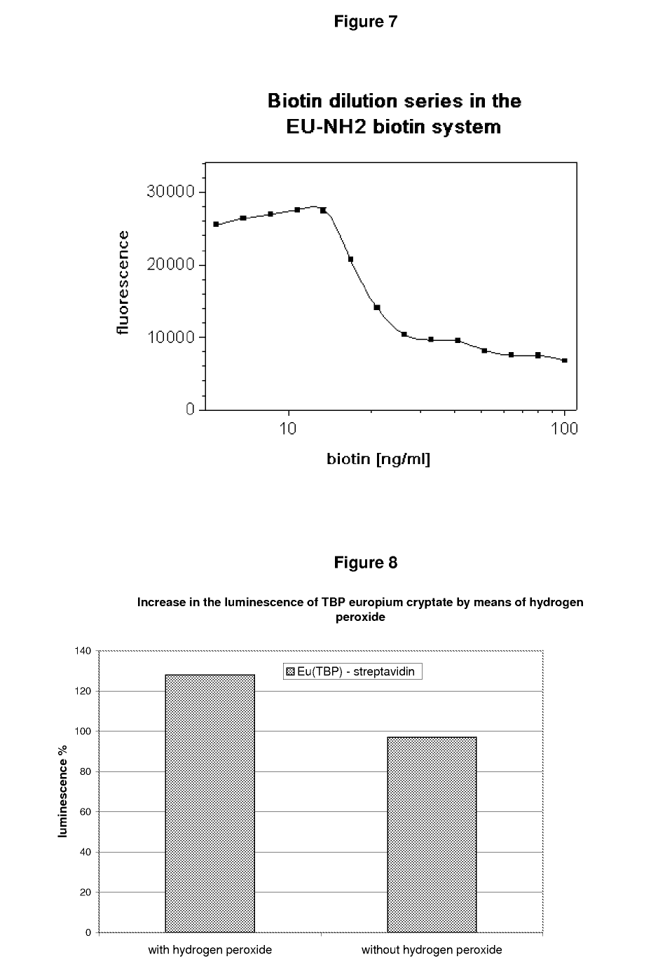

Streptavidin could be displaced from its bond to Eu—NH2-bitoin by free biotin. Thus, G24-BA9 could bind the fluorophore which led to the fluorescence being quenched. In view of the materials used (in particularly in view of the primitiveness of the conjugate used), the assay is surprisingly sensitive (see FIG. 7).

### Example 4

### Experiments with Respect to Protein Europium Cryptate

### Objective:

Detection of the displacement of a monoclonal antibody which quenches the fluorescence of europium from its bond to a conjugate of protein (macromolecule) and fluorophore (europium cryptate) by means of antibodies specifically binding the protein in the conjugate.

### Model System:

A conjugate of europium cryptate (fluorophore) and peroxidase (POD) (analyte) was used. The monoclonal antibody G24-BA9 quenching the fluorescence of europium cryptates was displaced from its bond to the fluorophore by the antibody binding peroxidase (POD). Said effect could be inversed by external addition of the free analyte (POD).

### Preparation of the Europium Cryptate/Peroxidase (POD) Conjugate Eu(NH₂)—POD

### Materials:

| | | |
|---|---|---|
| Cryptate NH₂ | (cisbio) | (cryptate NH₂ = TBP europium cryptate, see |
| | | page 17) |
| Peroxidase | (sigma) | |

### Coupling:

A reactive aldehyde group was generated at the sugar residues of the peroxidase and the POD thus activated was subsequently desalted against 0.1 M NaHCO₃. The thus prepared POD was mixed with cryptate NH₂ in an equimolar ratio. Coupling took place overnight at room temperature. Non-coupled aldehyde groups were blocked by means of ethanolamine, pH 8, and the conjugate was dialyzed against PBS.

The Eu(NH₂)—POD conjugate was used without further purification of the conjugate.

Displacement of G24-BA9 from its Bond to Eu(NH₂)—POD by Means of Anti-POD Sera

The following four components were mixed in measuring reactions of respectively 150 μl in the specified final concentrations:

| | | |
|---|---|---|
| Cryptate | Eu-NH₂-POD | dilution 1:2000 |
| Quencher | G24-BA9 | 5 μg/ml |
| Displacing antibody | anti POD sera | dilution 1:1000 |
| Analyte | POD | 20 μg/ml |

All components of the measuring sample were diluted in PBS-NKS (5% calf serum in PBS).

The fluorescence was measured after one hour in the cryptor (Cezanne). The excitation was caused by means of a nitrogen laser at 337 nm; the time resolved measurement of the fluorescence was carried out at 620 nm.

### Result:

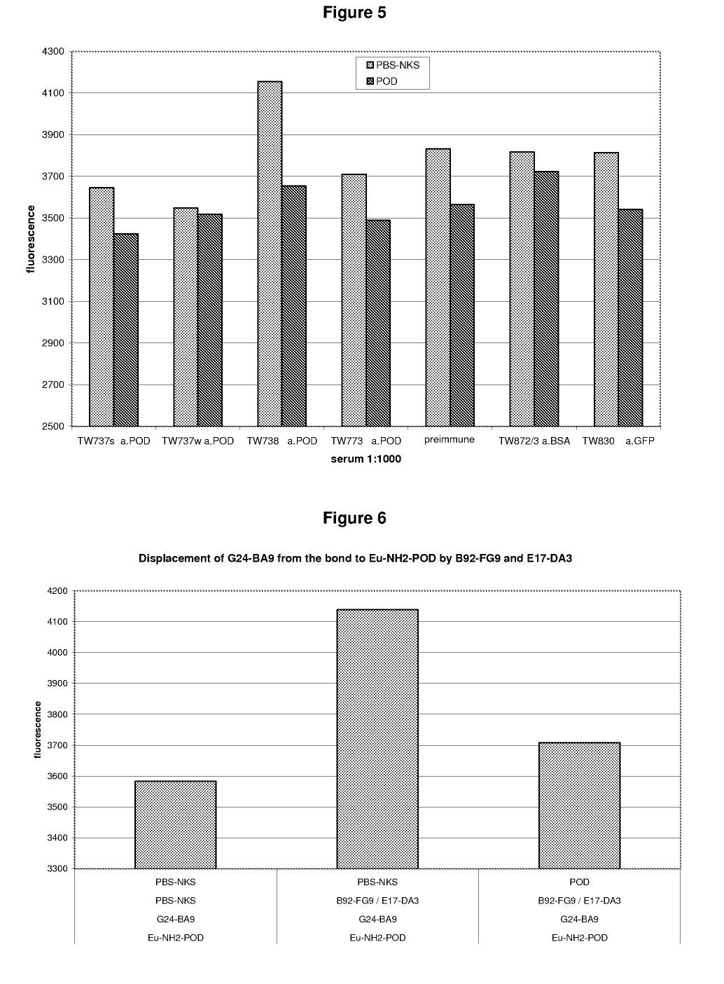

Of the total of four anti-POD sera, TW738 showed the desired effect. The polyclonal antibodies contained in the serum were partially capable of displacing G24-BA9 from its bond to Eu(NH₂)—POD. Said effect could be inversed by administration of the free analyte POD (see FIG. 5).

### Description of FIG. 5:

In the experiment, the sera of four test animals immunized with POD (peroxidase) were used (a.POD). A preimmune serum as well as an immune serum directed against BSA (bovine serum albumin) or respectively GFP (green fluorescent protein) were used as controls. The sera were diluted to 1:1000.

The luminescence of a Eu(NH₂)/POD conjugate was determined in the presence of the antibody G24-BA9 quenching the luminescence. Experiments were performed to test to which extent the antibodies binding POD contained in the immune sera (a.POD) were capable of displacing the antibody G24-BA9 from its bond to the conjugate. As a result of this displacement, an increase in the luminescence could be expected. The serum TW738 a.POD shows this effect. The other immune sera, as well as the preimmune serum were negative.

The effect observed in the serum TW738 a.POD could be inversed by administration of an excess of free POD. In that case, the POD-binding antibodies which were bound at the conjugate, mainly bound the free POD and not the conjugate. Thus, the conjugate bound by G24-BA9 and simultaneous quenching of the luminescence were observed.

Displacement of G24-BA9 from its Bond to Eu(NH₂)—POD by Means of Monoclonal Antibodies Binding POD

The following four components were mixed in measuring reactions of respectively 150 μl in the specified final concentrations:

| | | |
|---|---|---|
| Cryptate | Eu-NH₂-POD | dilution 1:2000 |
| Quencher | G24-BA9 | 5 μg/ml |
| Displacing antibody | anti-POD antibody | 20 μg/ml |
| Analyte | POD | 20 μg/ml |

All components of the measuring sample were diluted in PBS-NKS (5% calf serum in PBS).

The fluorescence was measured after one hour in the cryptor (Cezanne). The excitation was caused by means of a nitrogen laser at 337 nm; the time resolved measurement of the fluorescence was carried out at 620 nm.

### Result:

The desired effect could be observed on two of a total of 33 POD-binding monoclonal antibodies tested. Monoclonal antibodies B92-FG9 and E17-DA3 were able to partially displace G24-BA9 from its bond to Eu(NH₂)—POD. Said effect could be inversed by administration of the free analyte POD. The combination of both antibodies in one reaction proved to be particularly successful (see FIG. 6).

### Description of FIG. 6:

The two monoclonal antibodies B92-FG9 and E17-DA3 directed against POD were used in the experiment.

The luminescence of a Eu(NH₂)—POD conjugate was determined in the presence of the antibody G24-BA9 quenching the luminescence. Experiments were performed to test to which extent a mixture of the two antibodies binding POD was capable of displacing the antibody G24-BA9 from its bond to the conjugate. As a result of this displacement, an increase in the luminescence could be expected.

The first bar illustrates the luminescence without the antibodies binding POD. The second bar illustrates the ratios in the presence of the POD-binding antibodies. An increase in the luminescence is observed. The third bar illustrates that the addition of free POD inverses said effect and significantly reduces the luminescence.

### Example 5

### Increase in Luminescence Due to Hydrogen Peroxide

Increase in the luminescence of europium cryptates due to hydrogen peroxide.

TBP europium cryptate (Eu(TBP)-streptavidin, 10-8 M) and hydrogen peroxide (urea hydrogen peroxide, 10-5%) were used in the experiment. The measuring volume was 400 μl. Luminescence was measured in an ICCD (Andor) grid spectrometer.

As a result, the luminescence of the TBP europium cryptate was increased by 30% due to the addition of hydrogen peroxide (see FIG. 8).

### Example 6

### Experiments with Respect to Terbium Chelate

### Objective:

Detection of polyclonal antibodies quenching the fluorescence of terbium chelates in serum samples

### Model System:

Test animals (balb/c mice) were immunized with terbium chelate/OVA conjugates as described in example 2. Similarly, the same cryptate as in example 2 was used, with the difference that in this example Ln represents terbium [TbL].

### Preparation of the Terbium Chelate/Protein Conjugates

The terbium chelate [TbL] was present in form of NHS-ester. Coupling to the carrier proteins BSA (bovine serum albumin) and OVA took place overnight at room temperature in phosphate buffer, pH 7.4.

Subsequent to the coupling, the conjugate was dialyzed against PBS and used in the test without previous purification or characterization.

Immunization of the Test Animals with [TbL]-OVA and Serum Extraction

2 balb/C Mice were Immunized as Described in example 2.

Test of the Immune Sera for their Characteristic to Quench the Fluorescence of an [EuL]-BSA Conjugate

The measuring sample was composed of 75 μl of the diluted serum samples and 75 μl of [TbL]-BSA conjugate (dilution 1:10000; final concentration). All components of the measuring sample were diluted in PBS-NKS (5% calf serum in PBS).

The fluorescence was measured after one hour in the cryptor (Cezanne). The fluorescence of terbium chelate was determined at 545 nm.

### Result:

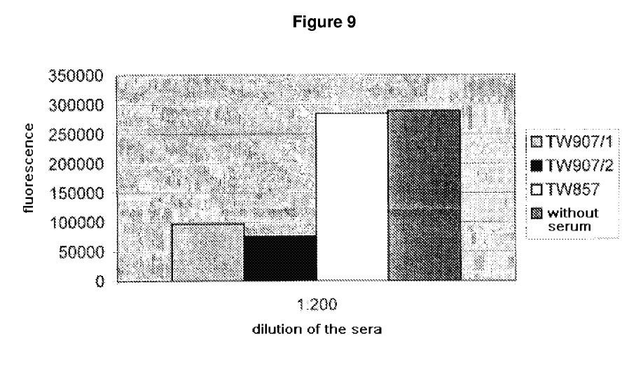

Both sera, TW907-1 (serum from the immunization 907-1) and TW907-2 (serum from the immunization 907-2) showed the desired fluorescence quenching effect, i.e. antibodies were present in the serum, which antibodies quenched (in analogy to the cryptate) the fluorescence of terbium chelates (see FIG. 9).

1. Method for the detection of one or more analytes, wherein said method comprises the following steps:
a) providing the following three components
a conjugate of the analyte and an europium cryptate fluorophore or a terbium cryptate fluorophore,
an antibody specific for the analyte, and
an antibody specific for the europium cryptate fluorophore or specific for the terbium cryptate fluorophore,
wherein the conjugate of the analyte and an europium cryptate fluorophore or
the conjugate of the analyte and a terbium cryptate fluorophore only allows for one single antibody to be bound,
b) addition of the analyte and
c) spectroscopic measurement of the fluorescence of the europium cryptate fluorophores or spectroscopic measurement of the fluorescence of the terbium cryptate fluorophores.

2. Method according to claim 1, further comprising hydrogen peroxide.

3. Method according to claim 1, further comprising at least one additional luminophore.

4. Method according to claim 1, wherein a covalent bond between an analyte molecule and a molecule of the europium cryptate fluorophore is present in the conjugate of analyte and europium cryptate fluorophore or wherein a covalent bond between an analyte molecule and a molecule of the terbium cryptate fluorophore is present in the conjugate of analyte and terbium cryptate fluorophore.

5. Method according to claim 1, wherein the analyte is selected from the group comprising:
pathogens, viruses, prions, bacteria, parasites, pharmaceuticals, antibiotics, cytostatics, psychoactive substances, narcotics, analgesics, cardiac drugs, metabolites, coagulation inhibitors, hormones, interleukins, cytokines, performance-enhancing drugs, drugs, toxins, noxious substances, pesticides, insecticides, wood preservatives, herbicides, fungicides, explosives, vitamins and flavors.

6. Method according to claim 1, wherein the ratio of antibody specific for the analyte and antibody specific for the europium cryptate fluorophore is defined and wherein the ratio of antibody specific for the analyte and antibody specific for the terbium cryptate fluorophore is defined.

7. Method according to claim 1, wherein the europium cryptate fluorophore or the terbium cryptate fluorophore comprises a tris-bipyridine cryptate.

8-11. (canceled)

12. A composition comprising
a) a conjugate of the analyte and an europium cryptate fluorophore,
wherein the conjugate of the analyte and an europium cryptate fluorophore only allows for one single antibody to be bound,
b) an antibody specific for the analyte and
c) an antibody specific for the europium cryptate fluorophore.

13. A composition comprising
a) a conjugate of the analyte and a terbium cryptate fluorophore,
wherein the conjugate of the analyte and a terbium cryptate fluorophore only allows for one single antibody to be bound,
b) an antibody specific for the analyte and
c) an antibody specific for the terbium cryptate fluorophore.

14. Composition according to claim 12, further comprising hydrogen peroxide.

15. Composition according to claim 12, further comprising at least one additional luminophore.

16. A kit for the detection of at least one analyte containing
a) a conjugate of the analyte and an europium cryptate fluorophore,
wherein the conjugate of the analyte and an europium cryptate fluorophore only allows for one single antibody to be bound,
b) an antibody specific for the analyte and
c) an antibody specific for the europium cryptate fluorophore.

17. A kit for the detection of at least one analyte containing
a) a conjugate of the analyte and a terbium cryptate fluorophore,
wherein the conjugate of the analyte and a terbium cryptate fluorophore only allows for one single antibody to be bound,
b) an antibody specific for the analyte and
c) an antibody specific for the terbium cryptate fluorophore.

18. Kit according to claim 16, further comprising hydrogen peroxide.

19. Kit according to claim 16, further comprising at least one additional luminophore.

20. Conjugates of the analyte and an europium cryptate fluorophore or conjugates of the analyte and a terbium cryptate fluorophore, wherein the analyte is selected from the group comprising:
pathogens, viruses, prions, bacteria, parasites, pharmaceuticals, antibiotics, cytostatics, psychoactive substances, narcotics, analgesics, cardiac drugs, metabolites, coagulation inhibitors, hormones, interleukins and cytokines, performance-enhancing drugs, drugs, toxins, noxious substances, pesticides, insecticides, wood preservatives, herbicides, fungicides, explosives, vitamins, flavors and the europium cryptate fluorophore or the terbium cryptate fluorophore comprises a tris-bipyridine cryptate.

21. Conjugates according to claim 20, wherein the analyte is covalently bound to the europium cryptate fluorophore or to the terbium cryptate fluorophore.

22. Use of the conjugates according to claim 20 for the detection of analytes selected from the group comprising:
pathogens, viruses, prions, bacteria, parasites, pharmaceuticals, antibiotics, cytostatics, psychoactive substances, narcotics, analgesics, cardiac drugs, metabolites, coagulation inhibitors, hormones, interleukins and cytokines, performance-enhancing drugs, drugs, toxins, noxious substances, pesticides, insecticides, wood preservatives, herbicides, fungicides, explosives, vitamins and flavors.

23. Method according to claim 2, wherein the ratio of antibody specific for the analyte and antibody specific for the europium cryptate fluorophore is defined and wherein the ratio of antibody specific for the analyte and antibody specific for the terbium cryptate fluorophore is defined.

24. Method according to claim 3, wherein the ratio of antibody specific for the analyte and antibody specific for the europium cryptate fluorophore is defined and wherein the ratio of antibody specific for the analyte and antibody specific for the terbium cryptate fluorophore is defined.

25. Method according to claim 4, wherein the ratio of antibody specific for the analyte and antibody specific for the europium cryptate fluorophore is defined and wherein the ratio of antibody specific for the analyte and antibody specific for the terbium cryptate fluorophore is defined.

26. Method according to claim 5, wherein the ratio of antibody specific for the analyte and antibody specific for the europium cryptate fluorophore is defined and wherein the ratio of antibody specific for the analyte and antibody specific for the terbium cryptate fluorophore is defined.

27. Method according to claim 2, wherein the europium cryptate fluorophore or the terbium cryptate fluorophore comprises a tris-bipyridine cryptate.

28. Method according to claim 3, wherein the europium cryptate fluorophore or the terbium cryptate fluorophore comprises a tris-bipyridine cryptate.

29. Method according to claim 4, wherein the europium cryptate fluorophore or the terbium cryptate fluorophore comprises a tris-bipyridine cryptate.

30. Method according to claim 5, wherein the europium cryptate fluorophore or the terbium cryptate fluorophore comprises a tris-bipyridine cryptate.

31. Composition according to claim 13, further comprising hydrogen peroxide.

32. Composition according to claim 13, further comprising at least one additional luminophore.

33. Kit according to claim 17, further comprising hydrogen peroxide.

34. Kit according to claim 17, further comprising at least one additional luminophore.

35. Use of the conjugates according to claim 21 for the detection of analytes selected from the group comprising:
pathogens, viruses, prions, bacteria, parasites, pharmaceuticals, antibiotics, cytostatics, psychoactive substances, narcotics, analgesics, cardiac drugs, metabolites, coagulation inhibitors, hormones, interleukins and cytokines, performance-enhancing drugs, drugs, toxins, noxious substances, pesticides, insecticides, wood preservatives, herbicides, fungicides, explosives, vitamins and flavors.
